(19) United States
(12) Patent Application Publication
(10) Pub. No.: US 2010/0215629 A1
(43) Pub. Date: Aug. 26, 2010

(54) TREATMENT OF TUMORS USING T LYMPHOCYTE PREPARATIONS

(75) Inventors: Jose Cohen, Paris (FR); David Klatzmann, Paris (FR); Sebastien Maury, Joinville Le Pont (FR); Francois Lemoine, Montrouge (FR)

Correspondence Address: OCCHIUTI ROHLICEK & TSAO, LLP, 10 FAWCETT STREET, CAMBRIDGE, MA, 02138, US

(73) Assignees: UNIVERSITE PIERRE ET MARIE CURIE (PARIS 6), Paris (FR); ASSISTANCE PUBLIQUE-HOPITAUX DE PARIS, Paris (FR); CENTRE NATIONAL DE LA RECHERCHE SCIENTIFIQUE, Paris (FR)

(21) Appl. No.: 12/682,646

(22) PCT Filed: Oct. 10, 2008
(86) PCT No.: PCT/FR2008/051842

§ 371 Date: Apr. 12, 2010

Related U.S. Application Data
(60) Provisional application No. 60/960,762, filed on Oct. 12, 2007.

(51) Int. Cl. A61K 35/12; A61P 35/00

(52) U.S. Cl. 424/93.71

(57) ABSTRACT

The invention relates to the treatment of a tumor in a patient, by injecting T lymphocytes depleted of regulatory T lymphocytes, and expressing a molecule allowing their specific destruction, the patient receiving beforehand a non-myeloablative or myeloablative lymphopenic treatment.

The present invention relates to the field of cell therapy of cancer. In particular, the invention relates to the treatment of malignant haemopathies, following a relapse or as a first line.

### TECHNOLOGICAL BACKGROUND OF THE INVENTION

The efficacy of the transplantation of allogenic haematopoietic stem cells (HSC) for the treatment of malignant haemopathies, including leukaemias, is based both on the myeloablation induced by conditioning but also on the transfer, within the transplant, of immunocompetent cells of the donor exerting an antileukaemic effect. This effect is called “graft versus leukaemia” (GVL). The existence of such a GVL effect was initially suggested by animal models and retrospective statistical analyses performed in humans comparing the levels of relapse of the leukaemia between the patients transplanted with an allogenic or syngenic donor, between those receiving a non-engineered or T-depleted transplant, and between those having developed, or not, an acute or chronic graft versus host (GVH) disease (Horowitz, et al. 1990). More recently, the demonstration of a GVL effect was provided by the transfusion of leukocytes obtained from peripheral blood of the donor in relapsed patients after the transplantation of allogenic HIC. Indeed, in patients exhibiting a relapse of chronic myeloid leukaemia (CML) after transplantation, it has been shown that the injection of leukocytes obtained from the initial donor (injection of lymphocytes of the donor or ILD) could induce a new remission, without requiring additional chemotherapy or radiotherapy (Kolb, et al 1990). This strategy has become a first-line treatment in these patients and has been developed for other recidivous haematological pathologies following the transplantation, such as myelodysplasias, lymphoblastic acute leukaemias (LAL) and myeloblastic acute leukaemias (MAL), certain lymphomas, or myelomas. Initially used following transplantation with a HLA genome-identical familial donor, ILD is today also used in the context of transplantations with a volunteer donor of non-related HSC (Porter, et al. 2000; Dazzi, et al. 2000a). In relapsed patients following transplantation, for whom there are few alternatives, and for whom prognosis is poor, ILD constituted an acceptable therapeutic option, for which improvements may nevertheless be expected.

Previously, several strategies were developed with the aim of amplifying the antitumor response following ILD.

The first, developed by the S. Slavin team, was aimed at stimulating the T lymphocytes of the donor either in vitro before injection or both in vitro but also in vivo following injection, with human recombinant interleukin 2 (IL-2) (Slavin, et al, 1996). Such an injection of “activated” T lymphocytes of the donor was reserved for patients not responding to an injection of fresh T lymphocytes of the donor. Accordingly, in 5 patients in a situation of cytological relapse of malignant haemopathy, the injection of activated T lymphocytes made it possible to obtain a complete remission of the haemopathy which had not been obtained after injection of fresh T lymphocytes of the donor. In this study, it was nevertheless difficult to attribute the efficacy of the second injection to the activation of the T lymphocytes since a beneficial cumulative effect of the injections of T lymphocytes of the donor may exist even in the fresh state (Dazzi et al, 2000b). It should also be noted that since 1996, this strategy of activation of the T lymphocytes with IL-2 has not been the subject of publications confirming its efficacy. Consequently, its use has not developed on a large scale.

Another strategy that is being developed aims to improve the presentation of tumor antigens by leukaemia cells and thereby make them sensitive to the GVL effect. It is based on the data obtained in vitro on myeloid leukaemia cells, which can be differentiated into dendritic cells with the aid of cytokines such as interleukin 4 or granulocyte monocyte colony stimulating factor (GM-CSF) (Brouwer, et al 2000; Chen, et al, 2000). The use of GM-CSF in vivo is thus envisaged for the period surrounding the injection of T lymphocytes of the donor in order to amplify the expected GVL effect (Kolb, et al, 2004).

Another strategy, proposed by Miller et al., 2007, consists of conditioning the patient with a lymphopenic chemotherapy, before an ILD. This conditioning led to an expansion of the lymphocytes with increased immune activation.

For their part, Powell et al. tried to administer autologous lymphocytes depleted of regulatory T lymphocytes of patients with a melanoma (Powell et al, 2007).

### SUMMARY OF THE INVENTION

The inventors now propose injecting T lymphocytes of the donor, combined with a myeloblastive or nonmyelobastive lymphopenic conditioning of the patient with the aim of increasing the antileukaemic effect of the lymphocytes injected.

More precisely, the subject of the invention is the use of T lymphocytes, depleted of regulatory T lymphocytes, and expressing a molecule allowing their specific destruction, for the preparation of a composition intended to treat a tumor in a patient, the composition being intended to be administered to the patient after a myeloablative or nonmyeloablative, preferably nonmyeloablative, lymphopenic treatment.

Also described is a method for treating a tumor, the method comprising:
conditioning the patient by a lymphopenic, preferably nonmyeloablative, treatment;
injecting into the patient T lymphocytes, which have been depleted beforehand of regulatory T lymphocytes, and which express a molecule allowing their specific destruction.

This procedure makes it possible to reduce immune rejection against the T lymphocytes, to increase the antileukaemic effect of the T lymphocytes injected, and to inject T lymphocytes obtained from a genetic pool different from that of the first donor and recipient.

Also described is a method for the first line treatment of a tumor, the method comprising:
conditioning the patient by a myeloablative or nonmyeloablative lymphopenic treatment;
injecting into the patient haematopoietic stem cells depleted of T lymphocytes; and
injecting into the patient T lymphocytes, which have been depleted beforehand of regulatory T lymphocytes, and which express a molecule allowing their specific destruction.

Depending on the cases, it is indeed possible to use autologous or allogenic T lymphocytes as described in detail below.

### DETAILED DESCRIPTION OF THE INVENTION

Definitions

The T lymphocytes are “depleted” or regulatory T lymphocytes, meaning that the T lymphocyte preparation administered to the patient comprises practically no regulatory T lymphocytes. Preferably, it comprises less than 10% of the regulatory T lymphocyte fraction before depletion, more preferably less than 1% of the regulatory T lymphocyte fraction before depletion.

The T lymphocytes express a “molecule allowing their specific destruction”. This may be a molecule encoded by a transgene or a molecule that is naturally expressed by the T lymphocytes, when the latter are allogenic. The term “specific destruction” means that only the T lymphocytes administered to the patient will be destroyed, to prevent the development of a GVH reaction.

The “molecule allowing their specific destruction” may be for example an antigen of the HLA system, the molecules Thy-1, NGF receptor or a truncated form of the receptor, or an antigen that is not immunogenic and not naturally expressed by the T lymphocytes. The T lymphocytes carrying either of these molecules can then be specifically destroyed by an antilymphocyte serum, or antibodies specifically directed against the said antigens.

The “molecule allowing the specific destruction” of the T lymphocytes may also be a molecule encoded by a “suicide gene”.

The term “suicide gene” refers to a gene encoding a molecule that is toxic for the cell expressing it, conditionally.

The T lymphocytes may be useful for treating the patient after an allotransplantation of haematopoietic stem cells, in particular in the case of a relapse.

The term “relapse” means that the tumor, which had shown a regression or a stagnation, has resumed its development or, where appropriate, has metastasized.

The T lymphocytes may also be administered as a first line, that is to say to treat tumors which were not previously treated in the patient by a transplantation of haematopoietic stem cells.

Source of the T Lymphocytes

The donor is preferably human, and may be a foetus, a newborn, a child, an adult.

The T lymphocytes preparations are obtained for example from peripheral blood, the blood product of a lymphapheresis, peripheral lymph nodes, the spleen, the thymus, cord blood, and the like.

Before being administered to the patient, the T lymphocytes may be engineered ex vivo so as to express a molecule allowing their specific destruction, after removal of the regulatory T lymphocytes. Preferably, the Treg lymphocytes are removed before introduction transduction of a gene encoding the molecule allowing the specific destruction of the T lymphocytes.

In the case of a treatment of a patient who has already received a transplant of haematopoietic stem cells, the T lymphocytes are preferably obtained from the first donor of haematopoietic stem cells. However, it is also possible that the T lymphocytes do not come either from the donor or from the recipient of the transplant of haematopoietic stem cells.

Depletion of Regulatory T Lymphocytes (Treg):

In 1995, the Shimon Sakaguchi group in Japan identified Treg lymphocytes as constituting a subpopulation of CD4+ T lymphocytes, representing about 5% of them, and they are characterized by a high and constitutive expression of the CD25 marker, which is the IL-2 receptor (Sakaguchi, et al., 1995).

More recent data show that human Treg repress the expression of CD127, which is the IL-7 receptor (Seddiki et al., 2006a). It has also been shown that Treg had a CD45RA+ naive cell phenotype (Seddiki et al., 2006b).

Advantageously, the depletion of regulatory T lymphocytes is obtained ex vivo by negative selection of the CD25+ cells or positive selection of the CD127+ cells.

The removal of the Treg cells may be carried out using affinity separation techniques, in particular magnetic separation, using antibody-coated magnetic beads, affinity chromatography, cytotoxic agents attached to a monoclonal antibody or used in combination with a monoclonal antibody. The separation techniques may use for example fluorescence-activated cell sorters.

The affinity reagents may be specific receptors or ligands for the markers indicated above. Preferably, they are antibodies, which may be conjugated for example with magnetic beads, with biotin, or with fluorochromes. Details on their possible separation techniques are presented in patent application US2006/0063256.

As in Attia et al., 2006, which removes the regulatory T lymphocytes on the basis of their expression of CD25 to increase the antitumor effect of an injection of T lymphocytes in the case of solid cancers, it is possible first of all to separate the CD4+ cells, and then among these the CD25+ cells.

In a particular embodiment, the donors undergo cytapheresis corresponding to two-three blood masses preferably using a Cobe Spectra machine. This sampling technique makes it possible to obtain on average 1 to 2×10¹⁰ mononuclear cells containing 20 to 30% of monocytes, 70 to 80% of T, B lymphocytes and NK cells. The cells are then freed of platelets and washed in a closed circuit in a COBE 2991 machine, thus making it possible to collect a cell population containing at least 5×10⁹ T lymphocytes. The strategy of Treg lymphocyte depletion is performed using cells obtained either from fresh cytapheresis product or from frozen cytapheresis product, in particular when it was not possible to take the donors from one of the centres involved in the trial. The depletion of the Treg lymphocytes is based on the removal of the CD25-positive cells. For that, a kit for separating cells in a Miltenyi closed circuit is used. In short, the fresh or thawed cells are labelled with an anti-CD25 monoclonal antibody directly coupled with magnetic beads. The cells are then deposited on a purification column and separated by the CliniMACS apparatus. The positive fraction is enriched with CD25 cells, the negative fraction is depleted of CD25 cells. This procedure makes it possible to deplete, at more than 95%, the CD25+ cells while preserving a cell population containing more than 2×10⁹ T lymphocytes.

While a fraction of the cells is intended for the quality control tests, upon constituting a cell bank, the majority of cells are cultured again so as to be transduced, amplified in culture and selected before being packaged in bags so as to be injected into patients.

Transduction of Molecules Allowing the Specific Destruction of the T Lymphocytes:

In a specific embodiment, the T lymphocytes are modified so as to express a transgene encoding a molecule allowing the specific destruction of the said T lymphocytes.

Specifically, the transgene is a “suicide” gene. For example, it may be a gene which encodes a molecule capable of phosphorylating a nucleoside analogue to a monophosphate molecule, itself convertible by cellular enzymes to a triphosphate nucleotide that can be incorporated into nucleic acids during extension under the effect of polymerases, the effect being the interruption of chain extension. The said nucleotide analogue may be for example acyclovir or gancyclovir. The said molecule expressed by the “suicide” gene may be in particular thymidine kinase (TK) of the herpes simplex virus type 1.

The herpes simplex virus 1 thymidine kinase (HSV1-TK) is capable, when it is present in a sufficient concentration in the cells in question, of phosphorylating nucleotide analogues, such as a acyclovir (9-((2-hydroxyethoxy)methyl]guanine) or gancyclovir (9-[1,3-dihydroxy-2-propoxymethyl]guanine), to monophosphate molecules which are themselves convertible by cellular enzymes to triphosphate nucleotides which can be incorporated into nucleic acids during extension under the effect of polymerases within the said cells, the effect being the interruption of chain extension and the cell death which follows.

In case of GVH, the nucleotide analogue (for example gancyclovir) is then administered to the patient.

Use may be made of any suitable technique for transferring the transgene, in particular by in vitro infection of the corresponding cells with an amphotropic Moloney type pseudo-viral particle. These viral particles are produced by a so-called “packaging” cell line which will have been constructed beforehand. A packaging line is capable of manufacturing all the structural elements constituting a viral particle, but is incapable of introducing into viral particles undergoing maturation the viral RNAs produced by this cell line. Accordingly, these so-called packaging lines continuously manufacture empty viral particles.

The introduction of an appropriate genetic construct, which contains the recombinant DNA as defined above, allows these packaging lines to be introduced into the empty viral particles, thus producing pseudo-viral particles. These pseudo-viral particles are capable of infecting various target cells, which target calls vary according to the packaging line which was used at the outset. For example, if this packaging line is derived from a so-called amphotropic Moloney virus, the viral particles produced perfectly infect human haematopoietic cells.

The conventional techniques for the production of cell lines transformed by a retroviral vector (see for example Danos et al., 1988 and Markowitz et al., 1988), can be transposed to the production of lymphocytes. Likewise, the genetic transfer techniques using such systems (Kasid et al., 1990) can be applied to the transfer, in humans, of the cells as defined above.

According to a particular embodiment of the invention, the T lymphocytes may be genetically modified using a retroviral vector SFCMM-2 encoding the “suicide” gene for HSV-TK/Neo fusion, according to the transduction method described in Ciceri et al., 2007.

One example of a transduction protocol comprises a first phase of cell culture of 1 to 6 days in order to induce the activation and the setting in cycle of the cells, and then a second phase of retroviral infection of 24 hours. The activation and the setting in cycle of the lymphocytes is obtained within 1 to 6 days after activation by an anti-CD3 antibody in an RPMI medium containing 10% human serum and 600 IU/ml of human recombinant interleukin 2. The day before the infection, the culture medium is replaced. The infection itself is made with supernatant containing either pseudotyped retroviral particles with an envelope derived from the Gibbon leukaemia virus (GALV) or pseudotyped lentiviral particles with an enveloped derived from the VSV virus and containing the bicistronic retroviral vector Thy-1-IRES-HSV1-TK. After retroviral infection, the cells are again cultured before being selected.

The selection of the transduced lymphocytes may be carried out by virtue of the presence of the Thy-1 reporter gene which makes it possible to select the T lymphocytes transduced with the aid of immunomagnetic beads. Accordingly, the T lymphocytes are incubated in the presence of an anti-Thy-1 monoclonal antibody directly coupled with biotin. The cells are then incubated with microbeads coupled with streptavidin and then deposited on a magnetic column. The Thy-1 positive transduced cells are separated by an immunomagnetic technique. After immunomagnetic selection, a population of Thy-1 positive T lymphocytes that has been at least 90% purified is obtained. After selection, the lymphocytes are either injected into the recipient or frozen for subsequent use.

Phenotype and Functional Validation of the Cell Preparations:

Preferably, the T lymphocyte preparations are tested in order to verify their phenotype. Examples of such tests are described below.

a) Immunophenotype analysis: the cell populations are studied by flow cytometry before and after depletion of CD25 cells, before and after genetic modification, before and after culture, before and after selection of the transduced cells. For that, techniques of double, triple or even quadruple labelling of the cells with monoclonal antibodies are used. The percentages of B and NK cells, and monocytes are determined with the CD19, CD14, CD16, CD56, CD45 markers. The subpopulations of T lymphocytes are studied using the CD3, CD4, CD8, CD45RO, CD45RA, CD62L markers. The perecentage of Treg lymphocytes is evaluated on the CD25 marker or the FOXP3 or CD127 or CD45RA marker alone and in a combination of markers.
b) Functional analysis: the T lymphocytes are analysed from the functional point of view before and after each of the handling steps according to two methods:

Allogenic stimulation: The donor cells, depleted or not of Treg lymphocytes, are exposed to irradiated mononuclear cells of the recipient, and irradiated mononuclear cells obtained from a volunteer are used as positive control. Cell proliferation under these different conditions, evaluated by a test for tritiated thymidine incorporation, makes it possible to compare if the depletion of Treg lymphocytes induces in vitro an increase in alloreactivity. Under these allogenic stimulation conditions, it is also possible to study by flow cytometry the intracytoplasmic secretion of cytokines (gamma-IFN-, IL-2, IL-4, IL-10) by the T lymphocytes.

Response to the booster antigens: The donor cells, depleted or not of Treg lymphocytes, are cultured for 4 days in the presence of irradiated autologous dendritic cells loaded or not with various antigens such as the tetanus toxin, tuberculin (PPD) and/or candidin. The dendritic cells generated from the donor monocytes serve as presenting cells. The T lymphocyte proliferation in response to these booster antigens is evaluated by the tritiated thymidine incorporation test. It is thus possible to evaluate in vitro if the populations depleted of Treg lymphocytes have a better capacity or otherwise to respond to conventional booster antigens.

Patient

The intended patient is a human being, regardless of age and gender. When the patient is old, a transplantation of haematopoietic stem cells is not indicated. It will then be preferable to administer the T lymphocytes as a first line.

The patient has a tumor, in particular a cancerous tumor. This may be a solid tumor or a malignant haemopathy.

Among the solid tumors, there may be mentioned lung, skin, kidney, bladder, bone, liver, pancreatic, ovarian, breast, uterine, prostate, colon, colorectal, and head and neck cancers, and the like.

Among the malignant haemopathies, there may be mentioned acute leukaemia, myelodysplasia, or the lymphoproliferative syndrome (such as chronic lymphoid leukaemia, myeloma, lymphoma), myelodysplasias, acute lymphoblastic leukaemias (ALL) and acute myeloblastic leukaemias (AML), lymphomas, or a myeloma.

The patient may in particular suffer from a tumor relapse, for example a malignant haemopathy relapse.

In a particular embodiment, the patient has undergone an allotransplantation of haematopoietic stem cells. In this case, it is possible for the T lymphocytes administered not to come from the donor or the recipient of the transplant of haematopoietic stem cells.

Preferably, the prior allotransplantation of HSC is derived from a familial donor, preferably geno-identical HLA, or from a non-related volunteer donor. This may be a transplantation with myeloablative or non-myeloablative conditioning, and it may have been T-depleted or not.

The intended patient may exhibit a molecular, cytogenetic or cytological relapse of the haemopathy regardless of the date thereof after the transplantation.

The relapse criteria are defined according to the haemopathy treated.

For example, for an acute leukaemia (AML or ALL) and myelodysplasia:
persistence of blood blasts and/or excess of medullary blasts (>5%), and/or
in case of a residual disease that can be analysed from the molecular point of view: absence of reduction (by at least one log) of the molecular signal relative to the pre-ILD point, or reduction followed by an increase (of at least one log relative to the nadir).
in case of a residual disease that can be analysed from the cytogenetic point of view (conventional or FISH): absence of a reduction (of at least 50%) of the number of mitosis carrying the abnormality or abnormalities, or reduction followed by an increase (of at least 50% relative to the nadir).

For a myeloma:
Stability or increase in the monoclonal peak relative to the pre-ILD point.
Light chain myeloma: stability or increase in the parameters capable of being evaluated (bone lesions, proteinuria, medullary plasmocyte infiltration).
Absence of a reduction, reduction followed a rise, or appearance of a plasmocytic tumor.

For a chronic lymphoid leukaemia, lymphomas:
Stability or increase of the clone (evaluated by flow cytometry, molecular biology) relative to the pre-ILD point.
Absence of a reduction or a reduction followed a rise in the tumor syndrome (evaluated from a clinical and/or radiological point of view) relative to the pre-ILD point.

When they are intended to be administered to patients as a first line, the T lymphocytes may be autologous with respect to the patient. They then express a transgene, preferably a “suicide” gene, allowing their specific destruction. The T lymphocytes administered may also be allogenic. They may then express a transgene allowing their specific destruction, or may be destroyed by an antilymphocyte serum.

When they are intended to be administered to a patient who has already undergone an allotransplantation of haematopoietic stem cells, the T lymphocytes are allogenic, or are obtained from third parties (namely neither the patient nor the donor of the first transplantation). They may then express a transgene allowing their specific destruction, or may be destroyed by an antilymphocyte serum.

Conditioning of the Patient

Before the administration of the T lymphocytes, the patient receives a non-myeloablative lymphopenic treatment.

Preferably, the non-myeloablative lymphopenic treatment is applied 2 to 8 days before the administration of the T lymphocytes.

The patient may also receive a lymphopenic and myeloablative treatment, in which case they further receive a transplantation of haematopoietic stem cells plus the T lymphocyte preparation.

A review of this type of treatment is presented in Petersen, 2007.

The patient may be fully irradiated (“total body irradiation”, for example at 2Gy), and/or receive a chemotherapy based on cyclophosphamide, fludarabine, and/or endoxan. For example, Miller et al., 2007, describe a non-myeloablastive lymphopenic treatment, which corresponds to an IV injection of cyclophosphamide 50 mg/kg once at D-6 and D-5, and injection of fludarabine 25 mg/m² from D-6 to D-2 (D being the day of transplantation of the T lymphocytes). Powell et al., 2007 describe another protocol comprising the injection of fludarabine 25 mg/m² for 5 days and of endoxan 60 mg/kg/day for 2 days.

Example of Protocol

According to a particular embodiment, the composition to be injected into the patient to be treated comprises the modified T lymphocytes obtained by:
i. lymphapheresis of the donor;
ii. removal of the regulatory T lymphocytes
iii. transduction of a gene encoding a molecule allowing the specific destruction of the T lymphocytes,
iv. and then culture of the T lymphocytes thus modified for ten to 21 days.

More precisely, the following protocol may be applied:

Around D-15, the T lymphocytes are collected from the donor and the regulatory T lymphocytes are removed ex vivo.

Next, on the same day, activation is performed in vitro (for example OKT3/IL-2) for a period of a few hours to 6 days, and then the cells are infected in the presence of a viral (retro- or lentiviral) supernatant comprising the suicide gene encoding TK and a membrane selection gene, for example Thy-1.

Next, the T lymphocytes are cultured until the desired cell number is obtained (for example 15 days). The lymphocytes expressing the TK gene will be selected on the basis of the expression of the Thy-1 gene used as reporter gene.

At D-6, the lymphopenic treatment is administered to the patient.

At D0, the TK+Treg depleted T lymphocytes are injected at doses of between 2.10⁶ and 10⁸ CD3+/kg.

In the case of GVH, ganciclovir is administered regardless of the time post-injection of the donor T lymphocytes.

### BIBLIOGRAPHIC REFERENCES

Attia P, Powell D J Jr, Maker A V, Kreitman R J, Pastan I, Rosenberg S A.: J Immunother (1997). 2006 March-April;29(2):208-14. Selective elimination of human regulatory T lymphocytes in vitro with the recombinant immunotoxin LMB-2.

Brouwer, R E, van der Hoorn, M, Kluin-Nelemans, H C, van Zelderen-Bhola, S, Willemze, R, and Falkenburg, J H. 2000. The generation of dendritic-like cells with increased allostimulatory function from acute myeloid leukemia cells of various FAB subclasses. Hum Immunol. 61:565-574.

Ciceri F, Bonini C, Marktel S, Zappone E, Servida P, Bernardi M, Pescarollo A, Bondanza A, Peccatori J, Rossini S, Magnani Z, Salomoni M, Benati C, Ponzoni M, Callegaro L, Corradini P, Bregni M, Traversari C, Bordignon C. 2007 Blood. 109(11):4698-707. Antitumor effects of HSV-TK-engineered donor lymphocytes after allogenic stem-cell transplantation.

Chen, X, Regn, S, Raffegerst, S, Kolb, H J, and Roskrow, M. 2000. Interferon alpha in combination with GM-CSF induces the differentiation of leukaemic antigen-presenting cells that have the capacity to stimulate a specific anti-leukaemic cytotoxic T-cell response from patients with chronic myeloid leukaemia. Br J Haematol. 111:596-607.

Danos, O. and Mulligan R. C. 1988. Safe and efficient generation of recombinant retroviruses with amphotropic and ecotropic host ranges. Proc. Natl. Acad. Sci. USA 85, 6460-6464.

Dazzi, F, Szydlo, R M, Cross, N C, Craddock, C, Kaeda, J, Kanfer, E, Cwynarski, K, Olavarria, E, Yong, A, Apperley, J F, et al. 2000a. Durability of responses following donor lymphocyte infusions for patients who relapse after allogenic stem cell transplantation for chronic myeloid leukemia. Blood. 96:2712-2716.

Dazzi, F, Szydlo, R M, Craddock, C, Cross, N C, Kaeda, J, Chase, A, Olavarria, E, van Rhee, F, Kanfer, E, Apperley, J F, et al. 2000b. Comparison of single-dose and escalating-dose regimens of donor lymphocyte infusion for relapse after allografting for chronic myeloid leukemia. Blood. 95:67-71.

Horowitz, M M, Gale, R P, Sondel, P M, Goldman, J M, Kersey, J, Kolb, H J, Rimm, A A, Ringden, O, Rozman, C, Truitt, R L, et al. 1990. Graft-versus-leukemia reactions after bone marrow transplantation. Blood. 75:555-562.

Kasid, A., Morecki, S., Aebersold, P., Cornetta, K., Culver, K., Freeman, S., Director, E. Lotze, M. T., Blaese, R. M. Anderson., W. F., and Rosenberg, S. A. 1990. Human gene transfer: characterization of human tumor-infiltrating lymphocytes as vehicles for retroviral-mediated gene transfer in man. Proc. Natl. Sci. USA 87, 473-477.

Kolb, H J, Mittermuller, J, Clemm, C, Holler, E, Ledderose, G, Brehm, G, Heim, M, and Wilmanns, W. 1990. Donor leukocyte transfusions for treatment of recurrent chronic myelogenous leukemia in marrow transplant patients. Blood. 76:2462-2465.

Kolb, H J, Schmid, C, Barrett, A J, and Schendel, D J. 2004. Graft-versus-leukemia reactions in allogenic chimeras. Blood. 103:767-776.

Miller J S, Weisdorf D J, Burns L J, Slungaard A, Wagner J E, Verneris M R, Cooley S, Wangen R, Fautsch S K, Nicklow R, Defor T, Blazar B R. 2007 Blood. 110(7):2761-3, Lymphodepletion followed by donor lymphocyte infusion (DLI) causes significantly more acute graft-versus-host disease than DLI alone.

Markowitz, D., Goff, S., and Bank, A. 1988. Construction and use of a safe and efficient amphotropic packaging cell line. Virology 167:400-406.

Petersen 2007. Medical Bulletin, 54 :112-39.Alloreactivity of therapeutic principle in the treatment of hematologic malignancies.

Porter, D L, Collins, R H, Jr., Hardy, C, Kernan, N A, Drobyski, W R, Giralt, S, Flowers, M E, Casper, J, Leahey, A, Parker, P, et al. 2000. Treatment of relapsed leukemia after unrelated donor marrow transplantation with unrelated donor leukocyte infusions. Blood. 95:1214-1221.

Powell D J Jr, de Vries C R, Allen T, Ahmadzadeh M, Rosenberg S A. 2007 Inability to mediate prolonged reduction of regulatory T Cells after transfer of autologous CD25-depleted PBMC and interleukin-2 after lymphodepleting chemotherapy. J Immunother May-June;30(4):438-47.

Sakaguchi, S, Sakaguchi, N, Asano, M, Itoh, M, and Toda, M. 1995 Immunologic self-tolerance maintained by activated T cells expressing IL-2 receptor alpha-chains (CD25). Breakdown of a single mechanism of self-tolerance causes various autoimmune diseases. J Immunol. 155:1151-1164.

Seddiki N, Santner-Nanan B, Martinson J, Zaunders J, Sasson S, Landay A, Solomon M, Selby W, Alexander S I, Nanan R, Kelleher A, Fazekas de St Groth B.2006a. Expression of interleukin (IL)-2 and IL-7 receptors discriminates between human regulatory and activated T cells. J Exp Med. July 10;203(7):1693-700.

Seddiki N, Santner-Nanan B, Tangye S G, Alexander S I, Solomon M, Lee S, Nanan R, Fazekas de Saint Groth B.2006b. Persistence of naive CD45RA+ regulatory T cells in adult life. Blood. 107(7):2830-8.

Slavin, S, Naparstek, E, Nagler, A, Ackerstein, A, Samuel, S, Kapelushnik, J, Brautbar, C, and Or, R. 1996. Allogenic cell therapy with donor peripheral blood cells and recombinant human interleukin-2 to treat leukemia relapse after allogenic bone marrow transplantation. Blood. 87:2195-2204.

1-18. (canceled)

19. A method for treating a tumor in a patient, which method comprising the patient with T lymphocytes, depleted of regulatory T lymphocytes, and expressing a molecule allowing their specific destruction, the patient having received a lymphopenic treatment beforehand.

20. The method according to claim 19, wherein the tumor is a malignant haemopathy.

21. The method according to claim 20, wherein the malignant haemopathy is an acute leukaemia, a myelodysplasia or a lymphoproliferative syndrome.

22. The method according to claim 19, wherein the tumor is a solid tumor.

23. The method according to claim 19, wherein the T lymphocytes express a “suicide” gene allowing their specific destruction.

24. The method according to claim 23, in which said “suicide” gene encodes a molecule capable of reacting with a nucleoside analogue in order to lead to the death of the said T lymphocytes.

25. The method according to claim 24, wherein said molecule encoded by a “suicide” gene is a molecule capable of phosphorylating a nucleoside analogue to a monophosphate molecule, itself convertible by cellular enzymes to a triphosphate nucleotide that can be incorporated into nucleic acids during extension under the effect of polymerases, the effect being the interruption of chain extension.

26. The method according to claim 25, wherein said nucleoside analogue is acyclovir or gancyclovir.

27. The method according to claim 26, wherein said molecule encoded by the “suicide” gene is thymidine kinase of the herpes simplex virus type 1.

28. The method according to claim 19, wherein the patient is suffering from a cancer relapse, following an allotransplantation of haematopoietic stem cells.

29. The method according to claim 28, wherein said T lymphocytes are obtained from neither the donor nor the recipient of the haematopoietic stem cell allograft.

30. The method according to claim 19, in which the T lymphocytes are intended to be administered to the patient as a first line, the said patient not having undergone the transplantation of haematopoietic stem cells.

31. The method according to claim 30, wherein the T lymphocytes are autologous with respect to the patient and express a transgene allowing their specific destruction.

32. The method according to claim 19, wherein the T lymphocytes administered to the patent are allogenic T lymphocytes.

33. The method according to claim 19, wherein the depletion of regulatory T lymphocytes is performed ex vivo by negative selection of the CD25+ cells or positive selection of the CD127+ cells.

34. The method according to claim 19, wherein the lymphopenic treatment is non-myeloablative.

35. The method according to claim 34, wherein the lymphopenic treatment consists of an administration of cyclophosphamide, fludarabine and/or endoxan.

36. The method according to claim 19, wherein the T lymphocytes are administered 2 to 8 days after the non-myeloablative lymphopenic treatment.

37. The method of claim 19, wherein the T lymphocytes comprises modified T lymphocytes obtained by:
i. lymphapheresis of the donor;
ii. removal of the regulatory T lymphocytes
iii. transduction of a gene encoding a molecule allowing the specific destruction of the T lymphocytes,
iv. and then culture of the T lymphocytes thus modified for 10 to 21 days.
